(19) United States
(12) Patent Application Publication
(10) Pub. No.: US 2010/0144796 A1
(43) Pub. Date: Jun. 10, 2010

(54) NEW POLYMORPHS OF ETHYL 3-[(2-{[4-(HEXYLOXYCARBONYLAMINO-IMINO- METHYL)-PHENYLAMINO]-METHYL-1-METHYL-1H-BENZIMIDAZOLE-5-CARBONYL) -PYRIDIN-2-YL-AMINO]-PROPIONATE

(75) Inventors: Mihaela Pop, Amsterdam (NL); Jaroslaw Mazurek, Rijswijk (NL); Mimoun Lamkadmi, Rotterdam (NL)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: Boehringer Ingelheim Pharma GMBH & CO.KG, Ingelheim (DE)

(21) Appl. No.: 12/515,225

(22) PCT Filed: Nov. 15, 2007
(86) PCT No.: PCT/EP2007/062415

§ 371 Date: Dec. 15, 2009

(30) Foreign Application Priority Data
Nov. 16, 2006 (DE) 10 2006 054 005.0

(51) Int. Cl. A61K 31/4439; C07D 401/12

(52) U.S. Cl. 514/338; 546/273.4

(57) ABSTRACT

The invention relates to new polymorphs of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, the preparation thereof and the use thereof as pharmaceutical compositions.

The invention relates to new polymorphs of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, methods of preparing them and their use as medicaments. This active substance having the chemical formula

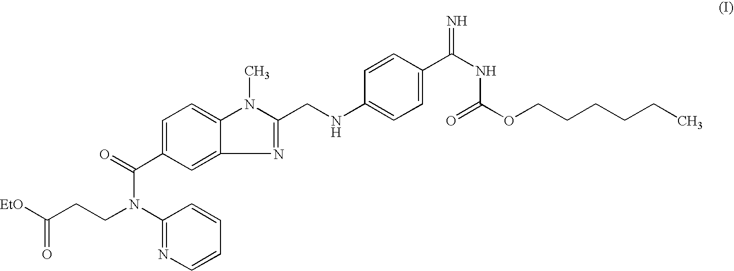

is already known from WO 98/37075, which discloses compounds with a thrombin-inhibiting effect and the effect of prolonging the thrombin time, under the name 1-methyl-2-[N-[4-(N-n-hexyloxycarbonylamidino)phenyl]-amino-methyl]-benzimidazole-5-yl-carboxylic acid-N-(2-pyridyl)-N-(2-ethoxycarbonylethyl)-amide. The compound of formula I is a double prodrug of the compound

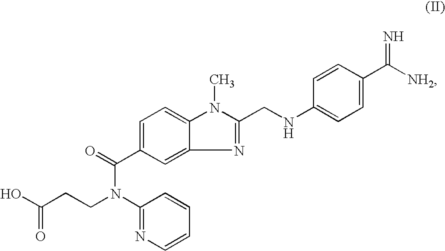

i.e. The compound of formula I is only converted into the active compound, namely the compound of formula II, after entering the body. The main indication for the compound of chemical formula I is the post-operative prevention of deep-vein thrombosis and the prevention of strokes.

The aim of the invention is to provide new polymorphs of the compound of formula I with advantageous properties for pharmaceutical use.

The abovementioned pharmacologically valuable properties of the disubstituted bicyclic heterocycles disclosed in the prior art constitute the basic prerequisite for effective use of the compounds as pharmaceutical compositions. However, to be permitted for use as a medicament, an active substance must also satisfy further requirements, besides actually being effective for the desired indication. These parameters are largely to do with the physicochemical nature of the active substance.

Without being restrictive, examples of these parameters are the stability of effect of the starting substance under various environmental conditions, the stability during production of the pharmaceutical formulation and stability in the final compositions of the drug. The pharmaceutically active substance used to prepare the pharmaceutical compositions should therefore have great stability which is ensured even under different environmental conditions. This is absolutely essential to prevent pharmaceutical compositions being used which contain breakdown products, for example, in addition to the active substance itself. In such a case the content of active substance present in the pharmaceutical formulation might be lower than specified.

The absorption of moisture reduces the content of pharmaceutically active substance as a result of the increased weight caused by the uptake of water. Pharmaceutical compositions with a tendency to absorb moisture have to be protected from moisture during storage, e.g. by the addition of suitable drying agents or by storing the drug in an environment where it is protected from moisture. In addition, the uptake of moisture may reduce the content of pharmaceutically active substance during manufacture if the pharmaceutical substance is exposed to the environment without being protected from moisture in any way. Preferably, therefore, a pharmaceutically active substance should be only slightly hygroscopic.

As the crystal modification of an active substance is important to the reproducible active substance content of a preparation, there is a need to clarify as far as possible any existing polymorphism of an active substance present in crystalline form. If there are different polymorphism modifications of an active substance care must be taken to ensure that the crystalline modification of the substance does not change in the pharmaceutical preparation later produced from it. Otherwise, this could have a harmful effect on the reproducible potency of the drug.

Another criterion which may be of exceptional importance under certain circumstances depending on the choice of formulation or the choice of manufacturing process is the solubility of the active substance. If for example pharmaceutical solutions are prepared (e.g. for infusions) it is essential that the active substance should be sufficiently soluble in physiologically acceptable solvents. It is also very important for drugs which are to be taken orally that the active substance should be sufficiently soluble.

The problem of the present invention is to provide a pharmaceutically active substance which not only is characterised by high pharmacological potency but also satisfies the above-mentioned physicochemical requirements as far as possible.

Surprisingly, it has been found that the novel polymorphs of the compound of formula I (dabigatran etexilate) solve this problem and have advantageous properties.

The invention therefore relates to the polymorphs of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate known as anhydrous form III, anhydrous form IV, monohydrate I, monohydrate II and solvate I. The invention further relates to pharmaceutical compositions containing at least of one of the above mentioned polymorphs as well as methods of preparing pharmaceutical compositions that are suitable for the prevention of venous thrombosis and stroke and contain the polymorphs according to the invention.

In a first aspect the present invention therefore relates to the five above-mentioned polymorphic forms of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, preferably in crystalline form, characterised by melting points of T_{mp.}=128±3° C. (anhydrous form III), T_{mp.}=133±3° C. (anhydrous form IV) or T_{mp.}=128±5° C. (monohydrate I) and T_{mp.}=128±5° C. (monohydrate II) as well as T_{mp.}=123±5° C. (solvate I) (determined by DSC; evaluated by peak maximum; heating rate: 10° C./min).

The DSC diagram of the anhydrous form IV is characterised in that three further weakly endothermic signals can be observed at approx. 59, 78 and 104° C. These signals can be put down to fully reversible solid-solid phase transitions, i.e. in the temperature range between 59-78, 78-104 and 104-128° C. there are three further high temperature phases of the anhydrous form IV.

The DSC diagram of the monohydrate II is characterised in that a further endothermic signal can be observed at approx. 100° C. This signal can be put down to the dehydration of the monohydrate II, as in a thermogravimetric experiment carried out parallel thereto the release of approx. 2.5% water is observed in this temperature range.

The DSC diagram of the solvate I is characterised in that a further endothermic signal can be observed at approx. 100° C. This signal can be put down to the desolvation of the solvate I, as in a thermogravimetric experiment carried out parallel thereto the release of approx. 7.3% nitrobenzene is observed in this temperature range.

The melting points of monohydrate I, monohydrate II and the anhydrous form III are substantially identical. This can probably be explained by the fact that both hydrates are dewatered on heating to produce the anhydrous form III and then the same value is measured as melting point for all three forms.

The invention further relates to methods of selectively producing the five polymorphic forms as well as the modifications that can be obtained by these methods.

According to the invention the anhydrous form III of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate is obtained by
a) dissolving ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base in a solvent mixture of diisopropylether/THF=60:40 at 60° C. with a concentration of approx. 120 mg/ml,
b) cooling the solution to a temperature of approx. 25° C. at a cooling rate of 1° C./h and leaving it to stand for a further 72 h at this temperature,
c) isolating the precipitated crystals, and
d) drying the product thus obtained at ambient temperature.

According to the invention the anhydrous form IV of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate is obtained by
a) dissolving ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base in 1,4-dioxane at ambient temperature with a concentration of approx. 120 mg/ml,
b) leaving the solution to equilibrate for 2 h with stirring at ambient temperature and then filtering it,
c) adding hexane (as antisolvent) to the clear solution at ambient temperature in a molar ratio to 1,4-dioxane of 1:1,
d) suction filtering the precipitated crystals and
e) drying the product thus obtained at ambient temperature.

According to the invention the monohydrate I of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate is obtained by
a) dissolving ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base in a solvent mixture of DMSO/cyclohexanone=80:20 at 60° C. with a concentration of approx. 120 mg/ml,
b) cooling the solution at a cooling rate of 1° C./h to a temperature of approx. 5° C. and leaving it to stand for a further 24 h at this temperature,
c) isolating the precipitated crystals and
d) drying the product thus obtained at ambient temperature.

According to the invention the monohydrate II of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate is obtained by
a) dissolving ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base in a solvent mixture of acetonitrile/acetone=60:40 at 60° C. with a concentration of approx. 60 mg/ml,
b) cooling the solution to a temperature of approx. 5° C. at a cooling rate of 1° C./h and leaving it to stand for a further 24 h at this temperature,
c) isolating the precipitated crystals and
d) drying the product thus obtained at ambient temperature.

According to the invention the solvate I (nitrobenzene) of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate is obtained by
a) suspending ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base in nitrobenzene at 50° C. with a concentration of approx. 333 mg/ml,
b) filtering the suspension while hot (50° C.) and cooling the resulting saturated solution at a cooling rate of 30° C./h to a temperature of approx. 20° C. and leaving it to stand for a further 24 h,
c) optionally, in the event that no crystals are formed within these 24 h, evaporating the solution down until crystallisation sets in,
d) isolating the precipitated crystals and
d) drying the product thus obtained at ambient temperature.

The crystalline forms of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate according to the invention were investigated more thoroughly by X-ray powder diffraction. The diagrams obtained are shown in FIGS. 1 to 5.

Tables 1 to 5 contain the data obtained in the analysis:

TABLE 1
X-ray powder reflections (up to 30 °2Θ) and intensities
(standardised) of ethyl 3-[(2-{[4-(hexyloxycarbonylamino-imino-
methyl)-phenylamino]-methyl}-1-methyl-1H-benzimidazole-5-carbonyl)-
pyridin-2-yl-amino]-propionate (anhydrous form III)

| 2 Θ [°] | d [Å] | I/Iₒ [%] |
|---|---|---|
| 5.13 | 17.22 | 2 |
| 8.87 | 9.96 | 72 |
| 10.64 | 8.31 | 30 |
| 11.31 | 7.82 | 21 |
| 11.79 | 7.50 | 40 |
| 12.84 | 6.89 | 21 |
| 13.87 | 6.38 | 7 |
| 14.34 | 6.17 | 10 |
| 15.19 | 5.83 | 11 |
| 15.91 | 5.57 | 100 |
| 16.68 | 5.31 | 35 |
| 17.71 | 5.00 | 2 |
| 18.38 | 4.82 | 9 |
| 19.41 | 4.57 | 35 |
| 20.34 | 4.36 | 66 |
| 21.36 | 4.16 | 19 |
| 22.31 | 3.98 | 9 |
| 23.57 | 3.77 | 24 |
| 23.92 | 3.72 | 26 |
| 24.84 | 3.58 | 20 |
| 25.39 | 3.50 | 85 |
| 26.73 | 3.33 | 31 |
| 27.41 | 3.25 | 18 |
| 28.10 | 3.17 | 16 |
| 28.99 | 3.08 | 11 |
| 29.37 | 3.04 | 15 |
| 30.05 | 2.97 | 7 |

Particularly characteristic peaks in the X-ray powder diffractogram of the anhydrous form III are d = 5.57 Å, d = 3.50 Å, d = 9.96 Å and d = 4.36 Å.

TABLE 2
X-ray powder reflections (up to 30 °2Θ) and intensities
(standardised) of ethyl 3-[(2-{[4-(hexyloxycarbonylamino-imino-
methyl)-phenylamino]-methyl}-1-methyl-1H-benzimidazole-5-carbonyl)-
pyridin-2-yl-amino]-propionate (anhydrous form IV)

| 2 Θ [°] | d [Å] | I/Iₒ [%] |
|---|---|---|
| 3.84 | 23.00 | 5 |
| 4.24 | 20.84 | 6 |
| 4.51 | 19.56 | 7 |
| 8.33 | 10.61 | 100 |
| 9.27 | 9.53 | 6 |
| 10.33 | 8.55 | 22 |
| 12.82 | 6.90 | 10 |
| 13.71 | 6.46 | 65 |
| 14.68 | 6.03 | 26 |
| 15.67 | 5.65 | 5 |
| 16.54 | 5.36 | 20 |
| 17.16 | 5.16 | 12 |
| 17.51 | 5.06 | 15 |
| 18.75 | 4.73 | 8 |
| 19.41 | 4.57 | 9 |
| 20.28 | 4.38 | 38 |
| 21.36 | 4.16 | 53 |
| 22.81 | 3.90 | 16 |
| 23.34 | 3.81 | 6 |
| 24.64 | 3.61 | 31 |
| 25.65 | 3.47 | 10 |
| 26.64 | 3.34 | 20 |
| 27.70 | 3.22 | 28 |

Particularly characteristic peaks in the X-ray powder diffractogram of the anhydrous form IV are d = 10.61 Å, d = 6.46 Å and d = 4.16 Å.

TABLE 3
X-ray powder reflections (up to 30 °2Θ) and intensities
(standardised) of ethyl 3-[(2-{[4-(hexyloxycarbonylamino-imino-
methyl)-phenylamino]-methyl}-1-methyl-1H-benzimidazole-5-carbonyl)-
pyridin-2-yl-amino]-propionate (monohydrate I)

| 2 Θ [°] | d [Å] | I/Iₒ [%] |
|---|---|---|
| 4.68 | 18.88 | 46 |
| 8.56 | 10.32 | 100 |
| 9.93 | 8.90 | 12 |
| 10.45 | 8.46 | 43 |
| 11.26 | 7.85 | 7 |
| 11.66 | 7.58 | 13 |
| 13.85 | 6.39 | 88 |
| 14.51 | 6.10 | 62 |
| 15.54 | 5.70 | 43 |
| 16.76 | 5.28 | 33 |
| 17.54 | 5.05 | 62 |
| 17.75 | 4.99 | 62 |
| 18.52 | 4.79 | 30 |
| 20.15 | 4.40 | 85 |
| 20.88 | 4.25 | 75 |
| 21.69 | 4.09 | 54 |
| 22.08 | 4.02 | 48 |
| 23.34 | 3.81 | 50 |
| 24.39 | 3.65 | 69 |
| 25.83 | 3.45 | 63 |
| 26.49 | 3.36 | 88 |
| 27.10 | 3.29 | 43 |
| 27.85 | 3.20 | 26 |
| 29.11 | 3.07 | 35 |

Particularly characteristic peaks in the X-ray powder diffractogram of the monohydrate I are d = 10.32 Å, d = 6.39 Å, d = 3.36 Å, d = 4.40 Å, d = 4.25 Å and d = 3.65 Å.

TABLE 4
X-ray powder reflections (up to 30 °2Θ) and intensities
(standardised) of ethyl 3-[(2-{[4-(hexyloxycarbonylamino-imino-
methyl)-phenylamino]-methyl}-1-methyl-1H-benzimidazole-5-carbonyl)-
pyridin-2-yl-amino]-propionate (monohydrate II)

| 2 Θ [°] | d [Å] | I/Iₒ [%] |
|---|---|---|
| 4.48 | 19.69 | 38 |
| 8.54 | 10.35 | 100 |
| 10.54 | 8.38 | 12 |
| 11.28 | 7.84 | 6 |
| 13.49 | 6.56 | 92 |
| 14.61 | 6.06 | 40 |
| 15.69 | 5.64 | 3 |
| 17.16 | 5.16 | 71 |
| 17.70 | 5.01 | 25 |
| 18.34 | 4.83 | 6 |
| 18.74 | 4.73 | 9 |
| 19.92 | 4.45 | 53 |
| 20.64 | 4.30 | 19 |
| 21.33 | 4.16 | 19 |
| 22.47 | 3.95 | 38 |
| 22.91 | 3.88 | 18 |
| 23.74 | 3.75 | 16 |
| 24.51 | 3.63 | 19 |
| 25.49 | 3.49 | 30 |
| 26.15 | 3.40 | 12 |
| 26.76 | 3.33 | 49 |
| 27.77 | 3.21 | 16 |
| 28.75 | 3.10 | 12 |
| 29.11 | 3.06 | 14 |
| 29.79 | 3.00 | 5 |

Particularly characteristic peaks in the X-ray powder diffractogram of the monohydrate II are d = 10.35 Å, d = 6.56 Å, d = 5.16 Å, d = 4.45 Å and d = 3.33 Å.

TABLE 5
X-ray powder reflections (up to 30 °2Θ) and intensities
(standardised) of ethyl 3-[(2-{[4-(hexyloxycarbonylamino-imino-
methyl)-phenylamino]-methyl}-1-methyl-1H-benzimidazole-5-carbonyl)-
pyridin-2-yl-amino]-propionate (solvate I with nitrobenzene)

| 2 Θ [°] | d [Å] | I/Iₒ [%] |
|---|---|---|
| 4.38 | 20.14 | 25 |
| 8.51 | 10.38 | 88 |
| 10.34 | 8.55 | 50 |
| 10.86 | 8.14 | 7 |
| 11.87 | 7.45 | 3 |
| 13.35 | 6.63 | 70 |
| 14.21 | 6.23 | 40 |
| 15.13 | 5.85 | 19 |
| 17.04 | 5.20 | 100 |
| 17.66 | 5.02 | 45 |
| 18.38 | 4.82 | 22 |
| 19.75 | 4.49 | 80 |
| 20.79 | 4.27 | 62 |
| 21.55 | 4.12 | 23 |
| 22.16 | 4.01 | 43 |
| 23.64 | 3.76 | 17 |
| 24.25 | 3.67 | 35 |
| 25.36 | 3.51 | 23 |
| 26.38 | 3.38 | 78 |
| 27.52 | 3.24 | 18 |
| 28.92 | 3.08 | 27 |

Particularly characteristic peaks in the X-ray powder diffractogram of the solvate I are d = 5.20 Å, d = 10.36 Å, d = 4.49 Å, d = 6.63 Å and d = 4.27 Å.

In the preceding Tables 1 to 5 the value “2 Θ[°” denotes the angle of diffraction in degrees and the value “d [Å]” denotes the specified distances in Å between the lattice planes.

The x-ray powder diagrams were recorded, within the scope of the present invention, using a Bruker GADDS diffractometer fitted with a Hi-Star Surface Detector and a Cu anode as the x-ray source (CuK_{α} radiation, λ=1.5418 Å, 40 kV, 40 mA).

### BRIEF DESCRIPTION OF THE FIGURES

FIGS. 1 to 5 show the X-ray powder diffractograms of the five crystalline forms of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate.

### EXAMPLES

The melting points were determined by DSC using an apparatus made by Mettler-Toledo (type: DSC 822e). The melting temperature used was the peak temperature of the corresponding melting peak in the DSC diagram. The accuracy of the melting points specified is about ±3° C., and in the case of the hydrates and the solvate±5° C., as the melting peaks of these forms are broader.

The starting compound ethyl 3-[(2-{[4-(amino-hexyloxycarbonylimino-methyl)-phenyl-amino]-methyl}-1-methyl-1H-benzimidazole-5-carbonyl)-pyridin-2-yl-amino]-propionate may for example be prepared as described in International Application WO 98/37075, Example 113.

### Example 1

### Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (anhydrous form III)

Approx. 1.0 g of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base (as described in WO 98/37075) are dissolved in 10 ml of a mixture of acetone/THF=80:20. 48 μl of this concentrated solution are placed in a well of a 96 well plate. The 96 well plate, filled accordingly, is placed in a vacuum chamber at 200 mbar and stored there for 6 days at ambient temperature. After the solvent has evaporated off the amorphous residue is combined with 40 μl of a mixture of diisopropylether/THF=60:40. The 96 well plate is then sealed and heated to 60° C. and kept at that temperature for 30 minutes. Then the plate is cooled to 25° C. at a cooling rate of 1° C./h. The plate is kept at this temperature for a further 72 h. The crystals formed are isolated by evaporation of the solvent in a vacuum chamber at 200 mbar.

### Example 2

### Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (anhydrous form IV)

Approx. 120 mg of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base (as described in WO 98/37075) are dissolved in 1 ml of 1,4-dioxane. The solution is equilibrated at ambient temperature for 2 h and then filtered to remove any undissolved matter. Approx. 1 ml of n-hexane as antisolvent is added to the clear solution. The precipitate formed is filtered off or centrifuged and dried at ambient temperature.

### Example 3

### Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (monohydrate I)

Approx. 1.0 g of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base (as described in WO 98/37075) are dissolved in 10 ml of a mixture of acetone/THF=80:20. 48 μl of this concentrated solution are placed in a well of a 96 well plate. The 96 well plate, filled accordingly, is placed in a vacuum chamber at 200 mbar and stored there for 6 days at ambient temperature. After the solvent has evaporated off the amorphous residue is combined with 40 μl of a mixture of DMSO/cyclohexanone=80:20. The 96 well plate is then sealed and heated to 60° C. and kept at that temperature for 30 minutes. Then the plate is cooled to 5° C. at a cooling rate of 1° C./h. The plate is kept at this temperature for a further 24 h. The crystals formed are isolated by evaporation of the solvent in a vacuum chamber at 200 mbar.

### Example 4

### Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (monohydrate II)

Approx. 1.0 g of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base (as described in WO 98/37075) are dissolved in 10 ml of a mixture of acetone/THF=80:20. 24 μl of this concentrated solution are placed in a well of a 96 well plate. The 96 well plate, filled accordingly, is placed in a vacuum chamber at 200 mbar and stored there for 6 days at ambient temperature. After the solvent has evaporated off the amorphous residue is combined with 40 μl of a mixture of acetonitrile/acetone=60:40. The 96 well plate is then sealed and heated to 60° C. and kept at that temperature for 30 minutes. Then the plate is cooled to 5° C. at a cooling rate of 1° C./h. The plate is kept at this temperature for a further 24 h. The crystals formed are isolated by evaporation of the solvent in a vacuum chamber at 200 mbar.

### Example 5

### Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (solvate I with nitrobenzene)

Approx. 665 mg of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base (as described in WO 98/37075) are added to 2 ml of nitrobenzene and heated to 50° C. The resulting suspension is filtered while hot at 50° C. The filtrate is brought to 20° C. at a cooling rate of 30° C./h and left to stand at this temperature for a further 24 h. Any crystals formed are isolated, or, if no crystals have yet formed, the solution is evaporated further until crystallisation takes place.

Examples of Formulations:

### Example 6

Dry ampoule containing 75 mg of active substance per 10 ml
Composition:

| | | |
|---|---|---|
| Active substance | 75.0 | mg |
| Mannitol | 50.0 | mg |
| water for injections | ad 10.0 | ml |

Preparation:

Active substance and mannitol are dissolved in water. After packaging the solution is freeze-dried. To produce the solution ready for use for injections, the product is dissolved in water.

### Example 7

Dry ampoule containing 35 mg of active substance per 2 ml
Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried.

To produce the solution ready for use for injections, the product is dissolved in water.

### Example 8

Tablet containing 50 mg of active substance
Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 9 mm.

### Example 9

Tablet containing 350 mg of active substance
Composition:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### Example 10

Capsules containing 50 mg of active substance
Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation:

(1) is triturated with (3). This trituration is added to the mixture of (2) and (4) with vigorous mixing.

This powder mixture is packed into size 3 hard gelatine capsules in a capsule filling machine.

### Example 11

Capsules containing 350 mg of active substance
Composition:

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

Preparation:

(1) is triturated with (3). This trituration is added to the mixture of (2) and (4) with vigorous mixing.

This powder mixture is packed into size 0 hard gelatine capsules in a capsule filling machine.

### Example 12

Suppositories containing 100 mg of active substance
1 suppository contains:

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

### Example 13

| | percentage composition | | | | | |
|---|---|---|---|---|---|---|
| | | insu- | active | | per | per |
| | core | lating | substance | | capsule | capsule |
| | material | layer | layer | total | [mg] | [mg] |
| tartaric acid | 61.3 | — | — | 61.3 | 176.7 | 353.4 |
| gum arabic | 3.1 | 2.8 | | 5.9 | 17.0 | 34.0 |
| talc | — | 5.6 | 3.2 | 8.8 | 25.4 | 50.7 |
| hydroxypropyl- | — | — | 4.0 | 4.0 | 11.5 | 23.1 |
| cellulose | | | | | | |
| active | — | — | 20.0 | 20.0 | 50.0 | 100.0 |
| substance | | | | | | |
| (based on | | | | | | |
| the base) | | | | | | |
| total | | | | 100.0 | 288.3 | 576.5 |

### Example 14

| | percentage composition | | | | | |
|---|---|---|---|---|---|---|
| | | insu- | active | | per | per |
| | core | lating | substance | | capsule | capsule |
| | material | layer | layer | total | [mg] | [mg] |
| tartaric acid | 38.5 | — | — | 38.5 | 55.5 | 166.5 |
| gum arabic | 1.9 | 1.7 | | 3.6 | 5.2 | 15.6 |
| talc | — | 3.5 | 6.4 | 9.9 | 14.3 | 42.8 |
| hydroxypropyl- | — | — | 8.0 | 8.0 | 11.5 | 34.6 |
| cellulose | | | | | | |
| active | — | — | 40.0 | 40.0 | 50.0 | 150.0 |
| substance | | | | | | |
| (based on | | | | | | |
| the base) | | | | | | |
| total | | | | 100.0 | 144.2 | 432.5 |

The preparation and structure of the pellets according to Examples 13 and 14 are described in detail in WO 03/074056.

1. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate in crystalline form, characterised by a melting point of T_{mp.}=128±3° C. (anhydrous form III) (determined by DSC; evaluated by peak maximum; heating rate: 10° C./min).

2. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate in crystalline form, characterised by a melting point of T_{mp.}=133±3° C. (anhydrous form IV) (determined by DSC; evaluated by peak maximum; heating rate: 10° C./min).

3. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate tetrahydrate in crystalline form, characterised by a melting point of T_{mp.}=128±5° C. with simultaneous release of the crystal water enclosed in the crystal lattice (monohydrate I) (determined by DSC; evaluated by peak maximum; heating rate: 10° C./min).

4. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate tetrahydrate in crystalline form, characterised by a melting point of T_{mp.}=128±5° C. with simultaneous release of the crystal water enclosed in the crystal lattice (monohydrate II) (determined by DSC; evaluated by peak maximum; heating rate: 10° C./min).

5. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate tetrahydrate in crystalline form, characterised by a melting point of T_{mp.}=123±5° C. with simultaneous release of the crystal water enclosed in the crystal lattice (solvate I) (determined by DSC; evaluated by peak maximum; heating rate: 10° C./min).

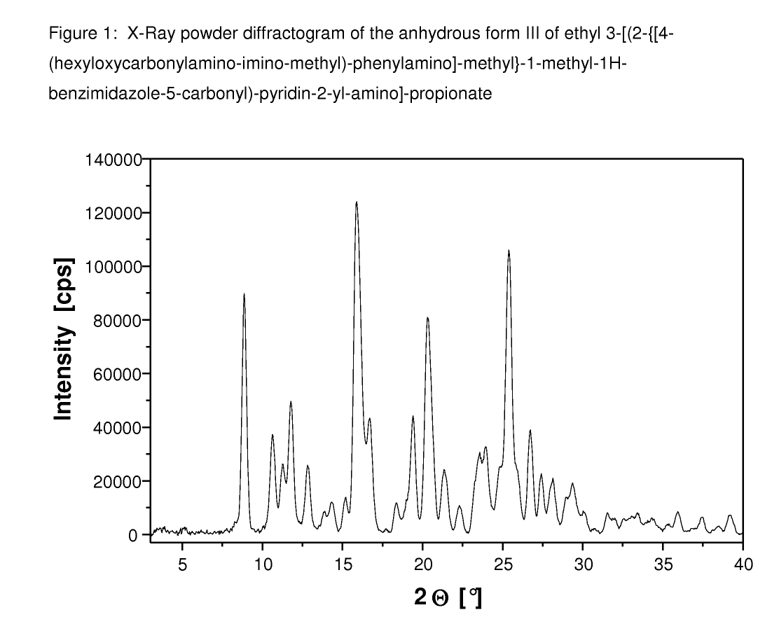

6. Compound according to claim 1, which has an X-ray powder diffractogram with the characteristic peaks shown in FIG. 1.

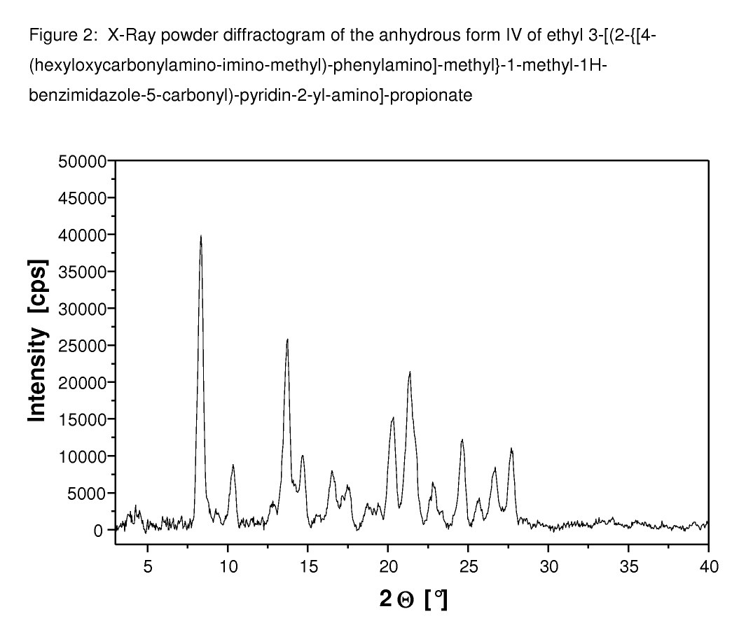

7. Compound according to claim 2, which has an X-ray powder diffractogram with the characteristic peaks shown in FIG. 2.

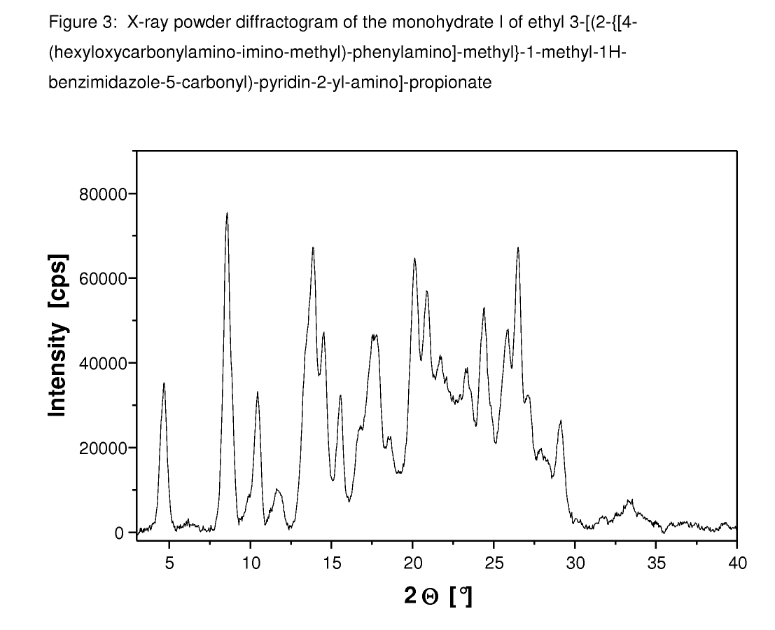

8. Compound according to claim 3, which has an X-ray powder diffractogram with the characteristic peaks shown in FIG. 3.

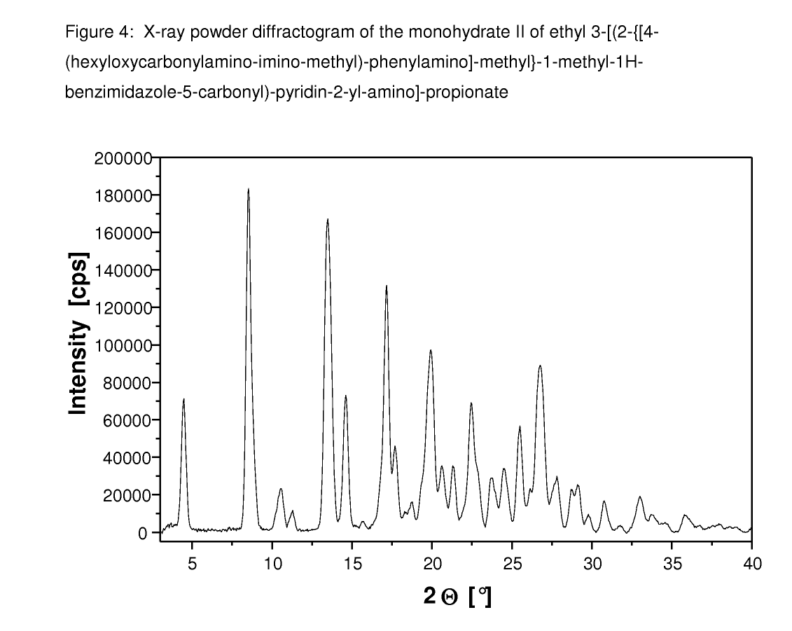

9. Compound according to claim 4, which has an X-ray powder diffractogram with the characteristic peaks shown in FIG. 4.

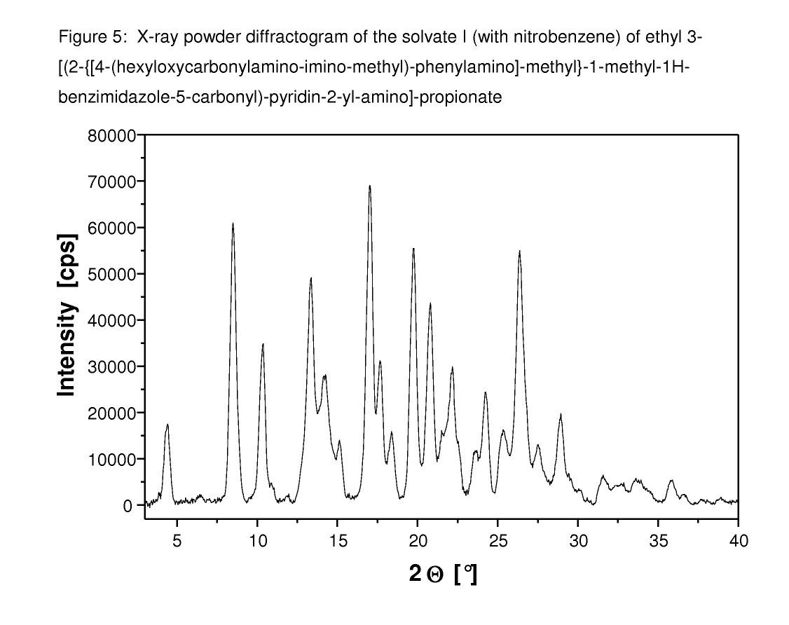

10. Compound according to claim 5, which has an X-ray powder diffractogram with the characteristic peaks shown in FIG. 5.

11. Compound according to claim 6, wherein the characteristic peaks in the X-ray powder diffractogram are d=5.57 Å, d=3.50 Å, d=9.96 Å and d=4.36 Å.

12. Compound according to claim 7, wherein the characteristic peaks in the X-ray powder diffractogram are d=10.61 Å, d=6.46 Å and d=4.16 Å.

13. Compound according to claim 8, wherein the characteristic peaks in the X-ray powder diffractogram are d=10.32 Å, d=6.39 Å, d=3.36 Å, d=4.40 Å, d=4.25 Åand d=3.65 Å.

14. Compound according to claim 9, wherein the characteristic peaks in the X-ray powder diffractogram are d=10.35 Å, d=6.56 Å, d=5.16 Å, d=4.45 Å and d=3.33 Å.

15. Compound according to claim 10, wherein the characteristic peaks in the X-ray powder diffractogram are d=5.20 Å, d=10.36 Å, d=4.49 Å, d=6.63 Å and d=4.27 Å.

16. Use of a compound according to claim 1 for preparing a pharmaceutical composition with the effect of prolonging the thrombin time.

17. Use of a compound according to claim 1 for preparing a pharmaceutical composition for the prevention of venous thromboses and stroke.

18. Pharmaceutical composition containing a compound according to claim 1 optionally together with one or more inert carriers and/or diluents.

19. Method of preparing a pharmaceutical composition characterised in that a compound according to claim 1 is incorporated in one or more inert carriers and/or diluents by a non-chemical method.

20. Process for preparing the anhydrous form III of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, characterised in that
a) ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base is dissolved in a solvent mixture of diisopropylether/THF=60:40 at 60° C. with a concentration of approx. 120 mg/ml,
b) the solution is cooled at a cooling rate of 1° C./h to a temperature of approx. 25° C. and left to stand for a further 72 h,
c) the precipitated crystals are suction filtered, and
d) the product thus obtained is dried at ambient temperature.

21. Process for preparing the anhydrous form IV of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, characterised in that
ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base is dissolved in 1,4-dioxane at ambient temperature with a concentration of approx. 120 mg/ml,
b) the solution is left to equilibrate for 2 h with stirring at ambient temperature and then filtered off,
c) hexane (as antisolvent) is added to the clear solution at ambient temperature in a molar ratio of 1:1 to 1,4-dioxane,
d) the precipitated crystals are suction filtered and
e) the product thus obtained is dried at ambient temperature.

22. Method of preparing the monohydrate I of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, characterised in that
a) ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base is dissolved in a solvent mixture of DMSO/cyclohexanone=80:20 at 60° C. with a concentration of approx. 120 mg/ml,
b) the solution is cooled at a cooling rate of 1° C./h to a temperature of approx. 5° C. and left to stand for a further 24 h at this temperature,
c) the precipitated crystals are suction filtered and
d) the product thus obtained is dried at ambient temperature.

23. Method of preparing the monohydrate II of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, characterised in that
a) ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base is dissolved in a solvent mixture of acetonitrile/acetone=60:40 at 60° C. with a concentration of approx. 60 mg/ml,
b) the solution is cooled at a cooling rate of 1° C./h to a temperature of approx. 5° C. and left to stand for a further 24 h at this temperature,
c) the precipitated crystals are suction filtered and
d) the product thus obtained is dried at ambient temperature.

24. Method of preparing the solvate I of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, characterised in that
a) ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base is suspended in nitrobenzene at 50° C. with a concentration of approx. 333 mg/ml,
b) the suspension is filtered hot (50° C.) and the resulting saturated solution is cooled to a temperature of approx. 20° C. at a cooling rate of 30° C./h to and left to stand for a further 24 h,
c) optionally, in the event that no crystals are formed within these 24 h, the solution is evaporated down until crystallisation sets in,
d) the precipitated crystals are suction filtered and
d) the product thus obtained is dried at ambient temperature.

25. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, anhydrous form III, obtainable by the method according to claim 20.

26. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, anhydrous form IV, obtainable by the method according to claim 21.

27. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate monohydrate I, obtainable by the method according to claim 22.

28. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate monohydrate II, obtainable by the method according to claim 23.

29. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate solvate I, obtainable by the method according to claim 24.
